(19) United States
(12) Patent Application Publication
(10) Pub. No.: US 2020/0206396 A1
(43) Pub. Date: Jul. 2, 2020

(54) DEVICE, METHOD AND USE FOR TRANSFUSING BODY FLUIDS

(71) Applicant: ORTHOGEN AG, Düsseldorf (DE)

(72) Inventors: Julien TROILLET, Markkleeberg (DE); Julio REINECKE, Köln (DE); Peter WEHLING, Düsseldorf (DE)

(73) Assignee: ORTHOGEN AG, Düsseldorf (DE)

(21) Appl. No.: 16/645,273

(22) PCT Filed: Sep. 14, 2018
(86) PCT No.: PCT/EP2018/074891

§ 371 Date: Mar. 6, 2020

(30) Foreign Application Priority Data
Sep. 15, 2017 (DE) 10 2017 008 657.5

(51) Int. Cl. A61M 1/02; B04B 5/04

(52) U.S. Cl. CPC A61M 1/02; B04B 5/0414

(57) ABSTRACT

The invention concerns a device for the transfusion of body fluid, comprising a chamber for accommodating the body fluid, wherein the chamber has a cylindrical section and narrows in its end region, wherein the inclination of the wall in the end region of the chamber is continuous along the longitudinal axis.

The present invention relates to a device, a method and a use in transfusing body fluids.

Manipulating body fluids in order to process them is known, in particular in a step of a method for centrifugation the body fluid. In this regard, the body fluid has to be withdrawn from the body and—optionally after processing, in particular centrifugation—transferred to another chamber or transfused back into the body. Syringes are known for the purposes of transfusing body fluids, in particular blood.

Centrifugation of body fluids which are in a syringe is known in order, for example, to separate out individual blood phases and to enable them to be provided for therapeutic purposes. In particular, the preparation of platelet-rich plasma (PRP) is very important in this regard.

During centrifugation, whole blood can be separated into individual components. The blood components can be separated from each other as a function of their density. As a rule, three phases are formed when whole blood undergoes centrifugation. The bottom layer with the highest density is constituted by the erythrocytes, while the top layer is the blood plasma which has been freed from cells. Between the top and the bottom layers is a thin layer formed by leukocytes and thrombocytes, what is known as the buffy coat. The buffy coat is of major interest because of the thrombocytes contained in it, because they are associated with the activation of many physiological processes. What is important here is that the buffy coat is a thin layer and has to be separated cleanly.

U.S. Pat. No. 8,052,969 B2 describes a method and a use of the method for the production of platelet-rich plasma using a 2-chamber syringe. In the 2-chamber syringe, the plunger of a first syringe is configured as a further syringe. After filling the first syringe, the entire 2-chamber syringe can undergo centrifugation, whereupon the individual components of the blood are separated as a function of their density. In a step which is subsequent to the centrifugation, the upper, platelet-rich plasma layer can be separated from the lower layer by means of the further syringe. In this regard, the plasma is aspirated into the cavity of the second syringe, whereby care must be taken to obtain a clean separation of the platelet-rich plasma from the higher density blood phase. Afterwards, the second syringe can be separated from the first syringe. The steps of the method that have to be carried out necessitate an experienced operator, but in doing this, contamination both from outside as well as with leukocytes or erythrocytes cannot be excluded. Thus, it is possible to lose or reduce the yield of PRP because separation of the leukocytes or erythrocytes is more difficult.

Against this background, the objective of the invention is to provide a device, a method and a use which enables individual components of body fluids to be provided with as small a loss of sample or contamination of the components of the body fluid as possible.

The objective is achieved by the subject matter of the independent patent claims. Advantageous embodiments are defined in the dependent patent claims and will become apparent from the description below.

The invention is based on the key concept that the shape of a chamber in which a processed body fluid is placed is configured in a manner such that the separation of the phases of a body fluid is facilitated, even for inexperienced operators. The shape of the chamber is selected in this regard such that the separation is inevitably facilitated during the transfusion of individual phases from the chamber, because during transfusion of the phases, an elongation occurs in order to minimize the boundary between the phases upon separation, and at the same time, the individual phases are transfused out of the chamber in a manner such that the separation of the individual phases which has already been carried out is not perturbed or affected. The shape of the chamber enables movement of the separated phases with as small a perturbance to the boundaries as possible.

The body fluid can be processed in the chamber in a centrifuge. The device is thereby suitable for being placed in a centrifuge. In this regard, the device may be configured to be processed in at least two sites or positions in the centrifuge. The two positions differ by a 180° rotation of the device transversely to the longitudinal axis of the device. This means that, for example with human or animal blood as the body fluid, the erythrocytes may be disposed at the “top” or “bottom” following centrifugation with respect to a pre-specified end of the device. Thus, for example, it is possible for the erythrocytes to be located adjacent to a pre-specified end or for the buffy coat with platelets and leukocytes to be at this end.

The invention provides a device for the transfusion of body fluid, comprising a chamber for accommodating the body fluid, wherein the chamber has a cylindrical section and narrows in its end region, wherein the inclination of the wall in the end region of the chamber is continuous along the longitudinal axis.

In a further aspect, the invention provides a device for the transfusion of body fluid, comprising a chamber for accommodating the body fluid, wherein the chamber has a cylindrical section and narrows in its end region. The inclination of the wall in the end region of the chamber along the longitudinal axis may be continuous or discontinuous. The aforementioned aspect of the invention may be combined with the subsequent cited features and further aspects of the invention.

The term “body fluid” in the context of the description encompasses elements of the human or animal body, the major components of which constitute a substance which is present in a liquid physical state. The term essentially comprises a fluid, however gaseous components should not be excluded. The term “body fluid” in particular encompasses saliva, lymph fluid, urine, bone marrow and, preferably, blood.

The term “transfusion” in the context of the description encompasses taking body fluid, in particular into a chamber, and subsequently dispensing at least portions of the body fluid, in particular from the chamber.

In the context of the description, the term “chamber” encompasses a cavity which can accommodate the body fluid. In this regard, the size of the cavity may be constant or variable, depending on the embodiment, wherein the cavity may have a maximum size and the size may be varied by moving a wall in order, for example, to force the fluid contained in it out of the chamber, or to aspirate fluid. In the context of the present description, a chamber may be sealed off from the environment, however, in particular after forming a sterile connection to the human or animal body, an open chamber may be employed in order to take up or dispense the body fluid.

The chamber may be pre-filled. As an example, the chamber may have a bioactive agent in one region. The bioactive agent may be a bioactive substance or a coating in a region of the chamber. The bioactive substance and/or the coating may be used for the modification of a body fluid located in or introduced into the chamber. In particular in the case of human or animal blood as the body fluid, suitable bioactive substances are anticoagulants including EDTA, citrate, heparin and/or derivatives thereof. The bioactive substances may also be substances which increase and/or modify the inner surface of the chamber. The substances which modify and/or increase the inner surface may have an inductive action for the formation of autologous proteins. The surface-modifying and/or surface-increasing substances include glass powder, glass granules, quartz powder, quartz sand, corundum, spherules, beads, sand and metals. The surface-modifying and/or surface-increasing substances also include organic compounds and polymers as well as biogenic or biological substances such as, for example, celluloses, collagens, alginates, nucleic acids and other proteins or metabolites formed by cells. The surface-modifying and/or surface-changing substance may have a substantially solid, liquid or gel-like consistency.

In the context of the description, the term “cylindrical section” encompasses a tubular section which is enclosed by a lateral surface and two further boundary surfaces which define cross sections. The “cylindrical section” may also be formed as a regular curve in a plane which is displaced over a predetermined distance along a straight line which is not contained in the aforementioned plane. The regular curve is one of the aforementioned cross sections at the start and end position of the displacement. The regular curve may in particular be a circle, an ellipse, a polygon or a combination of the aforementioned shapes. Particularly preferably in the context of the description, the cylindrical section is a perpendicular circular cylinder.

The term “wall” encompasses a boundary surface facing the inside of the chamber which, in particular in the direction of the longitudinal axis of the cylindrical section, may have the required shape for separation and/or transfusion.

The term “inclination of the wall” in the context of the description encompasses a variation in the distance of the wall from the central axis of a chamber with respect to the cylindrical section of the chamber. The inclination is considered and determined in respect of the profile in the direction of the central longitudinal axis. In particular, the mathematical inclination may be determined as a measure of the slope of a curve, wherein in one aspect of the invention, it is essential that the inclination of the wall should be continuous and thus in the mathematical context there are no steps, or a variation in the wall may be minor because the variations are sufficiently small in comparison to a distance along a longitudinal axis of the chamber. The inclination of the wall may in particular not have any discontinuities along the profile. The profile of the wall beyond the “cylindrical section” may preferably be an exponential, polynomially shaped and/or hyperbolic profile. A linear profile of the wall of the chamber beyond the “cylindrical section” is not excluded, as long as there is also another, further profile which may be exponential, polynomially shaped and/or hyperbolic.

The term “end region” of the chamber as used in the present description encompasses a region which directly adjoins the cylindrical section. In accordance with the present invention, a shape for the wall of a device is selected in a manner such that it can vary smoothly so that no disruption is caused when the body fluid flows inside the chamber. In particular, steps in the wall beyond the cylindrical section can be avoided. A constriction may be produced in order to reduce the boundaries between individual separated phases and to stretch out the individual volumes of the separated phases in the longitudinal direction. The body fluid can be transfused from the chamber, for example forced out of the chamber, with as little disruption as possible and with as accurate an observation of the location of the boundary between individual phases as possible. It is also possible for a portion of one phase to be forced out of the end region and/or aspirated out of it and/or in a subsequent step, for a portion of another or of the same phase to be forced out by means of cannulas or needles. In particular, the transition from the cylindrical section to the end region may be configured so as to be continuous, so that no steps are present in the inclination of the transition.

The end region may preferably be rotationally symmetrical with respect to the longitudinal axis of the device; here, a rotationally symmetrical configuration along the longitudinal axis of the device may have production engineering advantages. However, in a particularly preferred embodiment, the end region is not rotationally symmetrical to the longitudinal axis of the device. In particular, the opening in the end region may be off-centre with respect to the longitudinal axis of the device. In this regard, a suitable taper may be obtained wherein, for example when viewed in one direction, a wall may be observed which has an inclination that is lower or higher than an inclination of the wall transversely to the direction of view. In this manner, suitable conditions may be generated.

In a preferred embodiment, the length of the end region in the extent of the longitudinal axis is in the range from 18 mm to 24 mm, preferably 20 mm to 22 mm, more particularly preferably 21 mm. The volume of the end region in this regard is preferably in the range 4.1 mL±10%. The extent of the cylindrical section along the longitudinal axis in this regard is preferably 54 mm to 60 mm, particularly preferably 55 mm to 58 mm, more particularly preferably 57 mm, wherein the total length of the device for accommodating body fluid may be approximately 78 mm. The volume of the cylindrical section in the preferred exemplary embodiment is 30 mL±10%.

In a further embodiment, the extent of the end region along the longitudinal axis of the device may be 27 mm to 33 mm, preferably 29 mm to 31 mm, more particularly preferably 30 mm. The volume of the end region in this regard may be 5.6 mL±10%. The length of the extent of the cylindrical section along the longitudinal axis in this regard may be 44 mm to 50 mm, preferably 46 mm to 48 mm and more particularly preferably 47 mm, wherein the total extent of the device along the longitudinal axis may be approximately 77 mm. The volume of the cylindrical section may be 25.5 mL±10%.

In a preferred embodiment, a fluid volume in the chamber, with a base area which corresponds to a base of the cylindrical section, undergoes at least a 1.5 to 2.0-fold extension along the longitudinal axis of the chamber in the end region compared with the fluid volume in the cylindrical section. The term “base” of the cylindrical section encompasses a cross section which is produced when the cylindrical section is cut transversely to the longitudinal extent, or the base is the area which generates the cylindrical section by a parallel displacement of this area. In particular, the base in a perpendicular circular cylinder may be a circle. The base area of the fluid under consideration should correspond to the base in order to reflect the fact that the fluid volume under consideration in the chamber is a volume which covers at least the base of the cylindrical section and which extends along the longitudinal axis. It is tantamount to a reference volume as regards its spatial extent along the longitudinal axis between two “positions”, on the one hand when it is in the cylindrical section and on the other hand when it is in the region of the constriction.

In a preferred embodiment, the profile of the wall of the chamber in the end region is non-linear at least in sections, so that at least one section is present which is not straight. In this manner, an inclination which is as high as possible may be obtained for the wall.

In a preferred embodiment, the end region has an extent along the longitudinal axis of the chamber in the range from 15 mm to 40 mm and/or the cylindrical section has an extent along the longitudinal axis of the chamber in the range from 38 mm to 63 mm. In this manner, regions may be generated which allow for particularly good observation of the boundary between the individual phases. The longitudinal extent of the individual phases can be increased thereby, as long as the cross section of the corresponding region is also reduced.

In a preferred embodiment, the wall in the end region has (a) an exponential, (b) a polynomially shaped or (c) a hyperbolic profile, whereupon a continuous profile may be provided which does not contribute to any perturbances in the boundaries between the phases, but at the same time also allows for a reduction or constriction which, over a short distance of the longitudinal axis, leads to a large constriction. The profile of the wall in the end region does not have to be equally rotationally symmetrical about the longitudinal axis with respect to the longitudinal axis of the end region or the device, so that a non-rotationally symmetrical end region with respect to the longitudinal axis may also be provided. The opening in the end region may lie on the longitudinal axis of the device, but the opening at the end region may also be displaced with respect to the longitudinal axis.

In a preferred embodiment, the chamber has a rigid wall, whereupon a device that is easy to handle may be provided which can be held with the hands without disrupting the phases of the body fluid which have already been separated. Furthermore, a chamber with a rigid wall can be introduced into a centrifuge and, for example, be mounted on the rigid wall by means of a support in order to carry out the centrifugation.

In a preferred embodiment, an opening is provided in the end region of the chamber which is at a distance from the cylindrical section, and a connection piece is provided at the opening outside the chamber. In this manner, the body fluid can be poured into the chamber, and after processing is complete, in particular centrifugation, can be transfused from the chamber via the opening. The connection piece means that a connection of the chamber with a further chamber and/or with the human or animal body is simplified.

The term “connection piece” as used in the context of the present description describes a component which is present on the device in order to produce a connection of the device with external objects. Examples of the external objects are cannulas, infusion tubes, syringes and/or other chambers. A connection piece in the context of the present description may be configured as a part of a screw, Luer, Luer lock, bayonet and/or push connection.

In a preferred embodiment, the wall of the chamber is optically transparent, in at least one section of the end region, in order to ensure that viewing of the boundary moving through the chamber is facilitated. As an example, the chamber, in particular in the end region, may consist of a plastic which is configured so as to be transparent, in particular in a section of the end region. Plastics are easy to process during manufacture and are lightweight.

In a preferred embodiment, the chamber has a volume in the range from 5 to 70 mL, in order to accommodate a body fluid. Chambers with dimensions of this type can in particular be processed in a centrifuge, wherein the dimensions are also sufficient for a quantity of a phase of interest to be able to be obtained.

In particular, the device may be configured as a cavity in which a movable plunger can move. At the side of the cavity away from the plunger, the connection piece may be in the form of a Luer lock connection piece. The term “plunger” in the context of the description thereby encompasses a wall of the chamber which can be moved and guided into the cavity. At the movable wall, which essentially may correspond to the base area of the cylindrical section, a rod may be provided by means of which the wall can be displaced. In order to fill the cavity, the movable wall can be pulled with the rod in order to enlarge the effective cavity in which the body fluid can be accommodated. By means of the moving wall, the body fluid can be aspirated into the cavity. For the purposes of simplifying processing of the device in a centrifuge, a rod disposed on the movable wall may be separated from the wall or from a connecting element disposed on the wall. As an example, the rod may be broken off or unscrewed. In a particularly preferred embodiment, the device or chamber may be rotationally symmetrical with respect to the longitudinal axis.

In order to be able to move the body fluid or the plunger in the chamber properly, the inner wall of the cavity and/or the inner wall of the surface of the plunger contacting the cavity may be smooth in configuration. Furthermore, when the plunger and inner wall of the cavity cooperate, a particularly smooth movement may be executed, resulting in no disruption of the boundaries of the body fluid. In particular, the inner cavity and the plunger may be configured in a manner such that friction is minimized. This may be achieved by means of a suitable choice of material and/or by coating the relevant contact surface(s).

The invention also concerns a method for transfusing body fluid, comprising a chamber, wherein the body fluid is accommodated in the chamber and the chamber has a cylindrical section and narrows in an end region, wherein the inclination of the wall of the chamber in the end region is continuous along the longitudinal axis, and in particular a processed body fluid is allowed to flow out of the chamber via the end region.

The invention also concerns a use of a chamber during transfusion of a body fluid, wherein the chamber is used to accommodate the body fluid, wherein the chamber has a cylindrical section and narrows in its end region and wherein the inclination of the wall in the end region of the chamber is continuous along the longitudinal axis, wherein this chamber is in particular used to separate processed body fluid.

The invention will now be described in more detail with the aid of the drawings, which illustrate an embodiment of the invention. In the drawings:

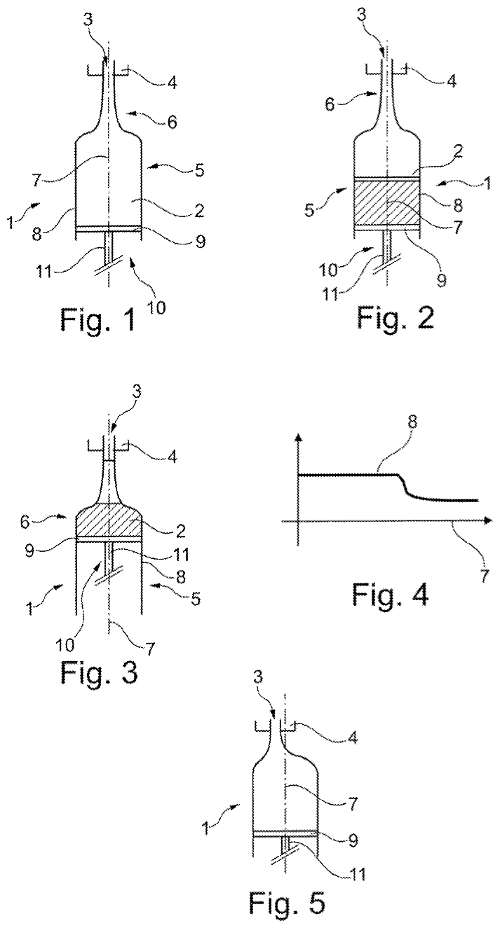

FIG. 1: shows a diagrammatic view of a device containing blood prior to centrifugation;

FIG. 2: shows a device in accordance with FIG. 1 following centrifugation;

FIG. 3: shows the device in accordance with FIG. 2 after transfusing a portion of the body fluid from the chamber;

FIG. 4: shows a profile of a wall along a longitudinal axis; and

FIG. 5: shows a further embodiment.

FIG. 1 shows a diagrammatic view of a device 1 containing blood. The device 1 has a chamber 2 for accommodating the blood. The device also has an opening 3 which is at the top in FIG. 1. By means of the opening 3, blood can be aspirated into the chamber 2 or can be discharged from the chamber 2. At the end of the device 1 adjacent to the opening 3 is a connection piece 4 which is formed as one piece with the device 1. In the case shown, the connection piece 4 is in the form of a Luer lock connection element.

The chamber 2 has a cylindrical section 5 and an end region which directly adjoins the cylindrical section 5, by means of which the cross section of the chamber narrows in the direction of the opening 3.

In the embodiment shown in FIG. 1, the cylindrical section 5 is configured as a straight circular cylinder and the chamber 2 is rotationally symmetrical in configuration. It is axially symmetrical and rotationally symmetrical with respect to the longitudinal axis 7. The wall of the chamber 2 is linear in the cylindrical section 5, and in the transition to the end region 6 as well as in the end region 6 itself, it is continuous without any steps. By considering the distance between the wall 8 of the chamber 2 and the longitudinal axis 7 as a function over the longitudinal axis 7 (see FIG. 4), the absence of steps in the profile of the wall 8 can then be described by stating that the inclination or slope of the function is continuous over the longitudinal axis 7.

Furthermore, the device 1 has a plate 9 which essentially has the dimensions of the base of the cylindrical section 5. The plate 9 is configured as a part of a plunger 10 with a rod 11. By moving the plate 9, the effective volume in the chamber 2 can be reduced or increased. As an example, the plate 9 may be drawn away from the opening 3 and blood can be aspirated into the chamber 2. In the exemplary embodiment shown in FIG. 1, the rod 11 may be separate from the plate 9 in order to allow for easy processing of the device 1 in a centrifuge. The longitudinal extent of the device 1 can be reduced by removing the rod 11 from the plate 9.

FIG. 2 shows the situation following centrifugation of whole blood. The upper layer or phase is the plasma and the lower layer has the erythrocytes. Between the plasma and the erythrocytes is what is known as the buffy coat, a thin layer formed from leukocytes and thrombocytes.

FIG. 3 shows the situation following movement of the plate 9 towards the opening 3 and a portion of the plasma having been forced out or aspirated from the chamber 2. Because of the continuous transitions and the continuous profile of the cylindrical section 5 and of the transition between the cylindrical section 5 and the end region 6 and the continuous profile in the end region 6 itself, there is almost no disruption to the boundaries between the individual phases and the narrowing of the end region 6 means that the volume is stretched out so that, as can be seen in FIG. 3, the buffy coat is extended in length and the boundary between the buffy coat and erythrocytes remains as a boundary which is as sharp as possible.

In the example shown in FIG. 3, removal is possible by means of a 10 mL syringe which can accommodate the buffy coat.

FIG. 4 diagrammatically shows the wall 8 of the chamber 2 both in the cylindrical section 5 as well as in the end region 6, as a function over the longitudinal axis 7. The wall profile is continuous, and in particular the inclination of the function has a continuous profile, whereupon a function is described thereby which does not have any steps and in particular does not have any discontinuities which could lead to a disruption of the boundary.

FIG. 5 diagrammatically shows a further embodiment of a device which is similar to the previously described embodiment of the device, but there is no rotational symmetry of the wall with respect to the longitudinal axis 7 in the end region 6.

1. A device (1) for the transfusion of body fluid, comprising a chamber (2) for accommodating the body fluid, wherein the chamber (2) has a cylindrical section (5) and narrows in its end region (6), wherein the inclination of the wall (8) in the end region of the chamber (2) is continuous along the longitudinal axis (7).

2. The device (1) as claimed in claim 1, wherein a fluid volume in the chamber (2), with a base area which corresponds to a base of the cylindrical section, undergoes at least a 1.5 to 2.0-fold extension along the longitudinal axis (7) of the chamber (2) in the end region (6) compared with a fluid volume in the cylindrical section (5).

3. The device (1) as claimed in claim 1 or claim 2, characterized in that at least in sections in the end region (6), the profile of the wall (8) of the chamber (2) is non-linear.

4. The device (1) as claimed in one of claims 1 to 3, characterized in that the end region (6) has an extent along the longitudinal axis (7) of the chamber (2) in the range from 15 mm to 40 mm and/or the cylindrical section (5) has an extent along the longitudinal axis (7) of the chamber (2) in the range from 38 mm to 63 mm.

5. The device (1) as claimed in one of claims 1 to 4, wherein the wall (8) in the end region (6) has (a) an exponential, (b) a polynomially shaped or (c) a hyperbolic profile.

6. The device (1) as claimed in one of claims 1 to 5, wherein the chamber (2) has a rigid wall (8).

7. The device (1) as claimed in one of claims 1 to 6, wherein an opening (3) is provided in the end region (6) of the chamber (2) and a connection piece (4) is provided at the opening (3) outside the chamber (2).

8. The device (1) as claimed in claim 7, wherein the connection piece (4) is configured as part of (a) a screw connection, (b) a Luer or Luer lock connection, (c) a bayonet connection and/or (d) a push connection.

9. The device (1) as claimed in one of claims 1 to 8, wherein the wall (8) of the chamber (2) is optically transparent in configuration, at least in a section of the end region (6).

10. The device (1) as claimed in one of claims 1 to 9, wherein the chamber (2) has a volume in the range from 5 to 50 ml.

11. A method for transfusing body fluid, comprising a chamber, wherein the body fluid is accommodated in the chamber, wherein the chamber has a cylindrical section and narrows in an end region, and wherein the inclination of the wall of the chamber in the end region is continuous along the longitudinal axis.

12. Use of a chamber during transfusion of a body fluid, wherein the chamber is used to accommodate the body fluid, wherein the chamber has a cylindrical section and narrows in its end region, and wherein the inclination of the wall in the end region of the chamber is continuous along the longitudinal axis.
